(19) United States
(12) Patent Application Publication
(10) Pub. No.: US 2014/0236002 A1
(43) Pub. Date: Aug. 21, 2014

(54) Methods and Systems for Imaging Tissue Motion Using Optical Coherence Tomography

(71) Applicant: UNIVERSITY OF WASHINGTON THROUGH ITS CENTER FOR COMMERCIALIZATION, Seattle, WA (US)

(72) Inventors: Ruikang Wang, Seattle, WA (US); Murray Johnstone, Bainbridge Island, WA (US); Tueng T. Shen, Woodinville, WA (US)

(73) Assignee: UNIVERSITY OF WASHINGTON THROUGH ITS CENTER FOR COMMERCIALIZATION, Seattle, WA (US)

(21) Appl. No.: 14/349,603

(22) PCT Filed: Oct. 17, 2012
(86) PCT No.: PCT/US2012/060593

§ 371 Date: Apr. 3, 2014

Related U.S. Application Data
(60) Provisional application No. 61/548,123, filed on Oct. 17, 2011.

(51) Int. Cl. A61B 3/10; A61B 5/0205; A61B 5/00

(52) U.S. Cl. CPC A61B 3/102; A61B 5/0066; A61B 5/0205; A61B 5/7235; A61B 5/7275
USPC 600/427

(57) ABSTRACT

A system and method for measuring tissue motion within a living tissue of the anterior segment and aqueous outflow system of the eye in a subject are provided. Tissue movements are extracted from a plurality of images acquired from the living tissue using an optical coherence tomography system. The images may be corrected using motion compensation. To extract the tissue movements from the images, waves from a cardiac pulse or other externally induced pulses from the subject are acquired, and a pulse wave is defined for a given time, which is then correlated with a velocity wave defined for a velocity of tissue and/or fluid movement within the tissue region for the same given time. Pulsatile motion is then isolated in the tissue region from the plurality of images.

### CROSS-REFERENCE TO RELATED APPLICATIONS

This application claims priority to U.S. Provisional Patent Application Ser. No. 61/548,123 filed on Oct. 17, 2011, which is hereby incorporated by reference in its entirety.

### BACKGROUND

Optical coherence technology (OCT) is a non-contact, noninvasive, real-time imaging modality that is capable of cross-sectional imaging of biological tissue with high spatial resolution. One example use of OCT is to image the anterior segment of the eye (AS-OCT) in vivo. Spectral domain OCT (SD-OCT) can also be used to create AS-OCT images.

Currently, OCT is used to provide static, structural images of a biological sample in vivo. Movements of tissues or fluids within the biological sample are difficult to monitor and measure, however. Because of this difficulty, current understanding of organs such as the eye, which contains the trabecular meshwork (TM) and specifically the aqueous outflow pathway that carries aqueous fluid, is limited. This limited understanding of physiologically important tissue movement that is important to normal function can hinder diagnosis and successful treatment of any problems in the tissue. An ability to characterize and/or image tissue motion is also important for quantitative assessment of the tissue biomechanical properties, changes in biomechanical properties as a result of disease processes and subsequent diagnosis, prognosis, or treatment of any issues or functional abnormalities associated with the tissue.

There is a need for a noncontact method and system for visualization of movement within a biological sample.

### SUMMARY

In accordance with the present invention, a system and a method are defined for measuring tissue motion within the aqueous outflow system in a living tissue in a subject. In one embodiment, the method may comprise extracting tissue movements from a plurality of images acquired from the living tissue of the aqueous outflow system and anterior segment of the eye using an OCT system. Extracting tissue movements from these tissues comprises acquiring pulse waves, in one embodiment induced by the cardiac pulse from the subject but may also be obtained by applying other external forces which alter intraocular pressure; correcting the acquired images using motion compensation, and determining, from the corrected images, a region for examination of the living tissue. Extracting tissue motion further comprises defining a velocity wave for a velocity within the region for examination for a given time, defining a pulse wave induced by the cardiac pulse or other pulse induced in the subject tissue for the given time, correlating the velocity wave and the pulse wave in the region for examination, and isolating pulsatile motion in the region for examination from the corrected acquired images.

In another embodiment, a system for measuring anterior segment and aqueous outflow system tissue motion in a living tissue is provided. The system comprises an OCT probe, an optical circulator, a coupler, a spectrometer, a digital pulsimeter, and a physical computer readable storage medium. The OCT probe, optical circulator, coupler, and spectrometer are used to acquire images of the living tissue. The physical computer readable storage medium comprises instructions executable to perform functions to extract tissue motion from the acquired images including acquiring waves induced by a cardiac pulse or other pulse waves induced in the subject tissue, correcting the acquired images using motion compensation, and determining, from the corrected images, a region for examination of the aqueous outflow system tissue. The functions further comprise extracting tissue movements, defining a velocity wave for a velocity within the region for examination for a given time, defining a pulse wave induced by the cardiac pulse or other induced pulse of the subject for the given time, correlating the velocity wave and the pulse wave in the region for examination, and isolating pulsatile motion in the region for examination from the corrected acquired images.

The system and method provide measurement of anterior segment and aqueous outflow system tissue motion in a living tissue, such as an ocular tissue, and may provide measurement of tissue motion within the outflow system of the anterior segment of the eye. The measurement of tissue motion may include a measurement of one or more types of surface or internal tissue displacement and changes over time; surface or internal tissue motion and changes over time; trabecular tissue compliance and changes over time; trabecular tissue elasticity and changes over time; trabecular tissue velocity of movement and changes over time; trabecular tissue excursions and changes over time; elastic modulus of trabecular tissue and changes over time; diameter and volume of the juxtacanalicular space and changes over time; measurement of diameter and volume of the intertrabecular spaces and changes over time; movement of cellular or collagenous structures at the entrances or within collector channel ostia and changes over time; Schlemm's canal diameter and volume and changes over time; collector channel ostia diameter and/or volume changes and changes over time; contour of the corneo-scleral junction and changes over time; angulation between the cornea and sclera at the corneoscleral junction and changes over time, diameter and/or volume of collector channels and changes over time; and scleral spur position, orientation, and changes over time.

The system and method may be used to diagnose, provide a prognosis, monitor treatment and guide treatment decisions for a disorder of the living tissues of the aqueous outflow system.

The system and method may be used for a subject at risk of any ocular disorder, including but not limited to an aqueous outflow pathway disorder. The aqueous flow pathway disorders may include any type of glaucoma, including but not limited to one or a combination of the following: open angle glaucoma, closed angle glaucoma, secondary glaucoma, pigmentary glaucoma, pseudoexfoliation glaucoma, uveitic glaucoma, neovascular glaucoma, low tension glaucoma and other glaucoma which either have a currently known or a currently unrecognized etiology. The system and method may be used to determine whether a subject is likely to respond to treatment of the aqueous outflow system, monitor the efficacy of treatment of the aqueous outflow system, make a treatment decision based on a prognosis related to use of the system and method to determine the functional status, guidance in medical, laser or surgical intervential decisions based on system and device-dependent measurements that provide information about the functional status of the outflow pathways and the likelihood of success of alternative interventions. Furthermore, the system and method may be used to determine the likely rate of progression of the disease associated with the ocular pathology.

The system and method may further comprise mapping the extracted tissue motion into the extracted microstructural images of the living tissue. The OCT system may be a phase sensitive OCT system, and the images may be acquired by applying light from a low coherence light source through an optical coupler that splits light from the light source to the tissue of interest and to a mirror, recombining light reflected from the tissue and the mirror through the optical coupler, and sending the recombined reflected light through a diffraction grating to a spectrometer. The light source may be any wavelength that is suitable for imaging the anterior segment of the eye. In one embodiment, the light source may have a central wavelength of about 850 nm. In another embodiment, the light source may have a central wavelength of about 1060 nm. In yet another embodiment, the light source may have a central wavelength of about 1310 nm.

These as well as other aspects and advantages of the synergy achieved by combining the various aspects of this technology, that while not previously disclosed, will become apparent to those of ordinary skill in the art by reading the following detailed description, with reference where appropriate to the accompanying drawings.

### BRIEF DESCRIPTION OF THE FIGURES

FIG. 1 depicts a schematic of an exemplary system in accordance with at least one embodiment;

FIG. 2a depicts a structural cross-section image of the corneo-scleral limbus, generated from the system of FIG. 1, in accordance with at least one embodiment;

FIG. 2b depicts a velocity cross-section image corresponding to the structural cross-section image of FIG. 2a, in accordance with at least one embodiment;

FIG. 2c depicts a graph illustrating an instantaneous velocity wave of the non-TM tissue plotted over time and the simultaneous pulse wave, in accordance with at least one embodiment;

FIG. 2d depicts a graph illustrating an instantaneous velocity wave of the TM tissue plotted over time and the simultaneous pulse wave, in accordance with at least one embodiment;

FIG. 2e depicts a graph illustrating exemplary power spectrums for each of the TM velocity wave and the digital pulse wave of FIG. 2d, in accordance with at least one embodiment;

FIG. 2f depicts a graph illustrating the significant correlation of the time instant between TM pulse peaks and the digital pulse peaks of the velocity wave and the digital pulse wave of FIG. 2d, in accordance with at least one embodiment;

FIG. 2g depicts a graph illustrating the significant correlation of the frequency components between the TM velocity wave and the digital pulse wave of the velocity wave and the digital pulse wave of FIG. 2d, in accordance with at least one embodiment;

FIG. 3a depicts an exemplary normalized strength mapping of the ocular tissue motion around the corneo-scleral limbus, in accordance with at least one embodiment;

FIG. 3b depicts the isolated TM strength mapping superimposed with a corresponding structural cross-sectional image generated from the system of FIG. 1, in accordance with at least one embodiment;

FIG. 3c depicts the isolated TM velocity cross-section superimposed with the structural cross-section image, corresponding to the downward velocity relative to the probe beam;

FIG. 3d depicts the isolated TM velocity cross-section superimposed with the structural cross-sectional image, corresponding to the upward velocity relative to the probe beam;

FIG. 4a depicts a schematic diagram of the microstructural components in the limbal area of the eye, in accordance with at least one embodiment;

FIG. 4b depicts a structural cross-sectional image of the human corneo-scleral limbus acquired from a temporal location generated from the system of FIG. 1, in accordance with at least one embodiment;

FIG. 4c depicts a structural cross-sectional image of the human corneo-scleral limbus after correction of geometrical distortions in FIG. 4b; and

FIG. 5 depicts a simplified flow diagram of an example method that may be carried out to measure motion in a living tissue, in accordance with at least one embodiment.

### DETAILED DESCRIPTION

In the following detailed description, reference is made to the accompanying figures, which form a part thereof. In the figures, similar symbols typically identify similar components, unless context dictates otherwise. The illustrative embodiments described in the detailed description, figures, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the figures, can be arranged, substituted, combined, separated, and designed in a wide variety of different configurations, all of which are explicitly contemplated herein.

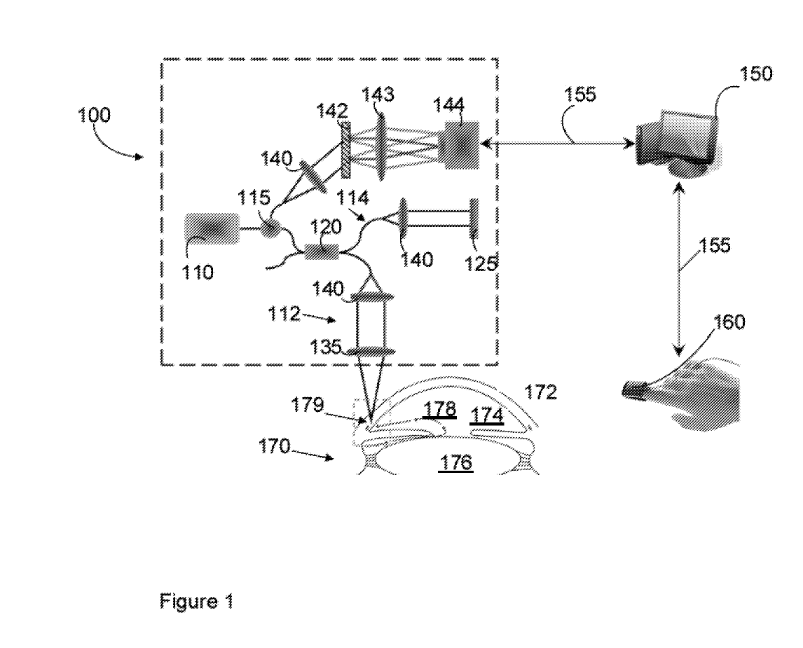

FIG. 1 depicts a schematic of an exemplary system 100 in accordance with at least one embodiment. The system may be used, among other things, to measure tissue motion within a living tissue sample of a subject. Thus, the system 100 may be used on a subject in vivo. As referenced herein, a subject may be a human subject.

In FIG. 1, an OCT system is shown as system 100. The system 100 may include a light source 110, a nonreciprocal optical element 115, a fiber coupler 120, a reference mirror 125, an objective lens 135, a plurality of collimating lenses 140, a diffraction grating 142, a focusing lens 143, and a spectrometer 144. The system 100 may further include a computing system 150 and a digital pulsimeter 160. A sample 170 to be imaged is also shown in FIG. 1. The OCT system may be a time-domain OCT system, spectral domain OCT (SD-OCT) system, or swept source OCT system.

The OCT system may be a phase-sensitive OCT (PhS-OCT) system. In one example embodiment, the OCT system may be an AS-OCT system based on a SD-OCT configuration. The OCT system may be integrated with the digital pulsimeter 160 as will be discussed in further detail below.

In one example embodiment, the light source 110 may be a low temporally coherent light source, such as a broadband superluminescent diode. In other embodiments, other light sources may be used. In one example embodiment, the light source 110 has a central wavelength within the range of about 850-1800 nm. The light source may have a central wavelength of about 850 nm, for example. In another example embodiment, the light source may have a central wavelength of about 1050 nm. In yet another example embodiment, the light source may have a central wavelength of about 1310 nm. In one example embodiment, the light source 110 has a spectral bandwidth of about 60 nm.

The nonreciprocal optical element 115 may be an optical circulator, and may have a first port connected to receive light from the light source 110. The nonreciprocal optical element 115 may further include a second port that may direct light from the first port to the fiber coupler 120 and receive light back from the fiber coupler 120, and a third port for directing light received from the fiber coupler 120 to the spectrometer 144.

The fiber coupler 120 serves as a beamsplitter, which transmits or splits some fraction of the power of the incident light power from the light source 110 into each of a sample arm 112 and a reference arm 114. Light returning from both the sample and the reference arms 112 and 114 may be fed to the spectrometer 144 via the nonreciprocal optical element 115. In one example embodiment, the fiber coupler 120 may comprise a pair of fibers partially fused together. The fiber coupler may be a 2×2 fiber coupler.

The reference mirror 125 serves to reflect light directed from the fiber coupler 120 back to the fiber coupler 120.

The fiber coupler 120 feeds light to a collimating lens 140 of the sample arm 112, which is then focused by the objective lens 135 onto the sample 170. In one example embodiment, the objective lens 135 may comprise a focal length of about 50 mm.

The diffraction grating 142 may serve to split and diffract light into several light beams that travel in different directions.

The focusing lens 143 may serve to focus the light beams received from the diffraction grating 142 into the spectrometer 144.

In one example embodiment, the spectrometer 144 may comprise a camera, such as an Indium Gallium Arsenide (InGaAs) line scan camera. The InGaAs line scan camera may provide scanning at about a 92 kHz A-line scan rate. The spectrometer 144 may have a spectral resolution of about 0.14 nm providing a measured imaging depth of about 3.0 mm in the air.

The spectrometer may send its output to the computing system 150 for further processing.

The digital pulsimeter 160 may be present to record a subject's digital pulse, through the subject's finger, for example. The digital pulse is significantly correlated to the pulsatile tissue motion for certain living tissues such as the TM, and may be used to aid in removing bulk motion from the tissue motion depicted in the OCT images, as will be described in further detail below. The digital pulse may be used as the time reference to characterize the pulsatile tissue motion.

The computing system 150 may include a processor, data storage, and logic. These elements may be coupled by a system or bus or other mechanism. The processor may include one or more general-purpose processors and/or dedicated processors, and may be configured to perform an analysis on the output from the spectrometer 144. An output interface may be configured to transmit output from the computing system to a display. The computing system 150 may be further configured to send trigger signals 155 to any of the spectrometer 144 and the digital pulsimeter 160. Trigger signals 155 may be sent by the computing system 150 to synchronize the OCT system with the digital pulse recording from the digital pulsimeter 160.

In operation, a subject is positioned at a designated location to allow for observation of desired biological tissues of the sample 170. In the example shown in the system 100, the sample 170 is an eye of a subject. The sample 170 comprises a cornea 172, an iris 174, a lens 176, a region comprising the aqueous outflow system and channels 178, and a box 179 depicting a region for examination. The sample 170 is observed in vivo in the example depicted in FIG. 1. The aqueous outflow system may be defined as including the trabecular meshwork, Schlemm's canal, the collector channel ostia, intrascleral collector channels, aqueous veins, and related recipient episcleral veins, in one example.

The light source 110 is directed through the nonreciprocal optical element 115 to the fiber coupler 120 which splits the light into the two arms 112 and 114, the reference arm 114 being directed at the reference mirror 125 and the sample arm 112 indicating the OCT probe beam being directed at the sample 170.

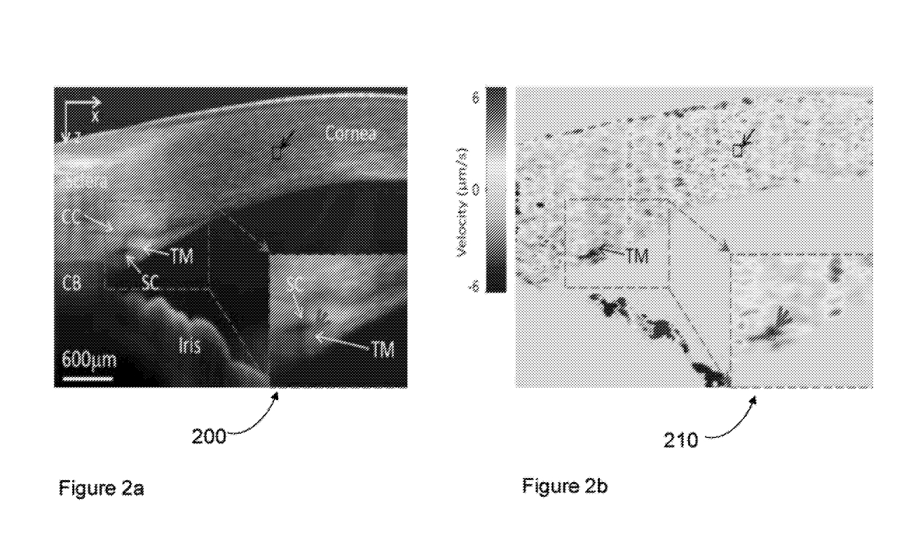

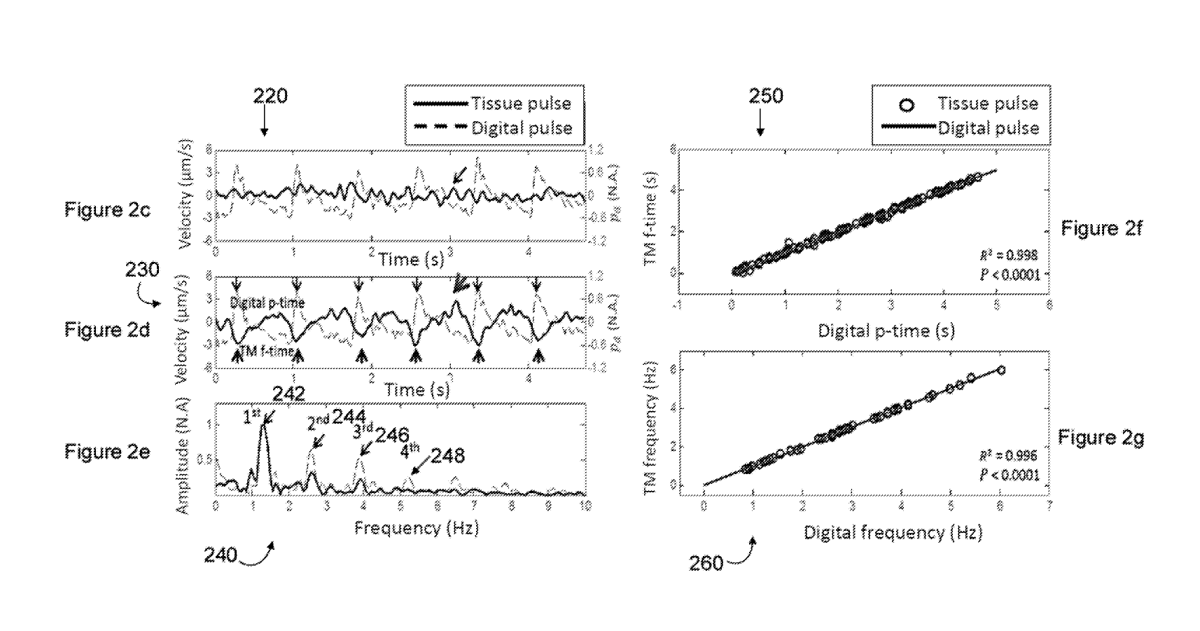

Light backscattered from the sample 170 in the sample arm 112 is then directed to the fiber coupler 120 and the nonreciprocal optical element 115, along with the reflected light from the reference mirror 125, which is then split via the diffraction grating and the various beams of light are then sent to the spectrometer 144. The spectrometer 144 may then feed the output to the computing system 150 for further processing, as will be described with reference to FIGS. 2a-4c.

In one example embodiment, for the sample 170 where the sample is a subject's eye, 1000 repeated OCT B-frames (e.g., time-lapse OCT B-frames, or MB-mode scan) may be captured at one spatial location around the corneo-scleral limbus of the anterior chamber of the eye, indicated by box 179 in FIG. 1, together with a simultaneous digital pulse. The 1000 B-frames may also be accomplished by taking, in the fast axis direction (i.e., B-scan), a frame rate of about 200 frames per second, and in the slow axis direction (i.e., C-scan), 200 sampling positions to capture one three-dimensional (3D) dataset, covering an approximate 4 mm distance, with five repeated B-frames at every position. Therefore, 1000 B-scans may be acquired to form a 3D data cube.

After the dataset has been acquired, an algorithm may be applied to the dataset to extract microstructural images.

Involuntary movement of a subject's eye and head creates a lateral and axial displacement between MB-mode B-frames. An algorithm may be used to correct the bulk tissue motion. The algorithm may include the use of cross-correlation between B-frames to calculate the frame shifts, from which all the B-frames are spatially aligned.

After motion compensation using the cross-correlation method, the phases of the OCT signals are calculated, and then the velocities of tissue motion Vₜ(x, z, t) are evaluated. To correct the phase shift calculation between B-frames, the velocity cross-sections Vₜ (x, z, t) may be corrected.

The instantaneous velocity wave Vₜ(t) may be defined as:

$${V}_{t} ( t ) = \frac{{\sum }_{l = - 1}^{1} {V}_{t} ( {x}_{0} + l , {z}_{0} + l , t )}{9} \qquad Equation 1$$

where l is an integer, (x₀, y₀) is a certain spatial location within the tissue of interest, and t is time.

The simultaneous digital/cardiac pulse wave P_{d}(t) may be resolved into a series of harmonic terms P_{di} (t) in the frequency domain, and may be defined as:

P_{d}(t)=P_{d0}+Σᵢ₌₁ⁿP_{di}(t) Equation 2

P_{di}(t)=a_{di} cos(2πift)+b_{di} sin(2πift) Equation 3

where the coefficients a_{di} and b_{di} are the amplitude of the cosine and sine wave components at a given harmonic frequency i*f, i is a positive integer (the harmonic number), and f is the fundamental frequency equivalent to the heart rate. P_{do} is the temporal mean value of the function. FIG. 2e illustrates exemplary results from using these functions. In FIG. 2d, the velocity is plotted over time. Both the digital p-time and the tissue f-time (in the example of FIG. 2d, the trabecular mesh (TM) is the tissue) are plotted.

Because most energy in an arterial pulse is contained in the first five harmonic components, the first five harmonic terms may be used, i.e., n=5.

The frequency contents of the tissue velocity wave and the digital/cardiac pulse wave may be correlated, and the instantaneous TM velocity wave Vₜ(t) may also be resolved into a series of harmonic terms Vₜᵢ(t) in a similar manner as the digital/cardiac pulse:

Vₜ(t)=Vₜ₀+Σᵢ₌₁ⁿVₜᵢ(t) Equation 4

Vₜᵢ(t)=aₜᵢ cos(2πift)+bₜᵢ sin(2πift) Equation 5

where the coefficients aₜᵢ and bₜᵢ are the amplitude of the cosine and sine wave components at a given harmonic frequency i*f. Vₜ₀ is the temporal mean value of the function, and again, n=5.

Finally, to facilitate the characterization of the tissue motion, the tissue pulsatile motion may be automatically isolated from the corrected tissue velocity images. To accomplish this, first, the motion strength is evaluated. To evaluate the motion strength, the coefficients aₜᵢ, bₜᵢ, and the magnitude Mₜᵢ were each calculated at the first three harmonic frequencies 1f, 2f, and 3f for each dataset using the heart rate f determined from the corresponding digital/cardiac pulse wave P_{d} (t) as follows:

$$\begin{matrix}{a}_{ti} = \frac{T}{2} {\int }_{- T / 2}^{T / 2} {V}_{t} ( t ) cos ( 2 π i f t ) d t \\ {b}_{ti} = \frac{T}{2} {\int }_{- T / 2}^{T / 2} {V}_{t} ( t ) sin ( 2 π i f t ) d t\end{matrix} ( for i = 1 , 2 , 3 , etc ) \qquad Equation 6$$

The magnitude Mₜᵢ of the TM velocity wave for each harmonic frequency can be expressed as:

Mₜᵢ=\sqrt{(aₜᵢ²+bₜᵢ²)} Equation 7

The tissue motion strength may then be defined as the normalized summation of the magnitude at the first two harmonic frequencies to map the tissue motion strength:

Sₜ=normalized(Mₜ₁+Mₜ₂) Equation 8

Next, the motion strength threshold is set and a motion mask may be generated by binarizing the motion strength map: setting 1/e-maximum as the threshold, where e=2.71828 is the natural constant.

Then, the tissue motion strength may be isolated from the motion strength map using the motion mask for spatially characterizing tissue motion.

Finally, the tissue velocity may be isolated from the corrected velocity images using the motion mask.

Harmonic analysis may thereafter be applied to quantify the dynamic pressure-flow relationship in the arterial and aqueous outflow systems. Because the pulsatile ocular tissue motion is induced by the pulsatile nature of ocular hemodynamics, the dynamic correlation between the ocular tissue motion and the ocular hemodynamics (such as IOP pulsation, flow or pressure pulsation in ocular arteries, cardiac pulsation) may be an important index with which to assess ocular physiology. The pulsatile ocular tissue motion is related to the digital/cardiac pulse by calculating the phase lag Δθᵢ between the digital pulse harmonic and the simultaneous tissue motion harmonic for each pair of a dataset:

Δθᵢ=θ_{di}−θₜᵢ Equation 9

where the phase of digital pulse wave θ_{di} and the phase of tissue pulse wave θₜᵢ for each harmonic frequency can be respectively calculated as:

θ_{di}=tan⁻¹(b_{di}/a_{di}), Equation 10

θₜᵢ=tan⁻¹(bₜᵢ/aₜᵢ) Equation 11

Accordingly, the time delay Δtᵢ between the digital pulse harmonic and the simultaneous tissue motion harmonic may be as follows:

$$Δ {t}_{i} = \frac{Δ {θ}_{i}}{2 π i f} \qquad Equation 12$$

All of the above-described calculations may be performed by a computing system such as the computing system 150. Statistical analysis software may be present on the computing system to perform the various calculations.

In another example embodiment, a correlation analysis may further be performed to assess the relationship of the phase lag Δθᵢ and time delay Δtᵢ with the heart rate and age of the subject. The results of correlation may be expressed as a P value for the hypothesis test and a R² value for the Pearson's linear correlation coefficient. Generally, the phase lag reduces as the heart rate and the age of a subject increases and the time delay generally reduces as the heart rate and the age of a subject increases.

The sample 170, as described above, may be a living ocular tissue, specifically in the anterior segment of the eye. In one example embodiment, the sample 170 may be the anterior segment of the eye and may provide measurement of tissue motion within the outflow system of the anterior segment of the eye. The measurement of tissue motion may include a measurement of one or more types of surface or internal tissue displacement and changes over time; surface or internal tissue motion and changes over time; trabecular tissue compliance and changes over time; trabecular tissue elasticity and changes over time; trabecular tissue velocity of movement and changes over time; trabecular tissue excursions and changes over time; elastic modulus of trabecular tissue and changes over time, diameter and volume of the juxtacanalicular space and changes over time; measurement of diameter and volume of the intertrabecular spaces and changes over time; movement of cellular or collagenous structures at the entrances or within collector channel ostia and changes over time; Schlemm's canal diameter and volume and changes over time; collector channel ostia diameter and/or volume changes and changes over time; contour of the corneo-scleral junction and changes over time; angulation between the cornea and sclera at the corneoscleral junction and changes over time, diameter and/or volume of collector channels and changes over time; scleral spur position, orientation, and changes over time.

The measurement of tissue motion may be used to diagnose, provide a prognosis, monitor treatment and guide treatment decisions for a disorder of the sample 170 of a subject. The treatment may include medical, laser, or surgical intervention. In one example embodiment, the measurement of tissue motion may determine whether the subject is at risk of an aqueous outflow pathway disorder or has ocular pathology that will result in that disorder, as well as providing a prognosis for likelihood of the subject to respond to treatment for the ocular pathology or monitoring the efficacy of treatment of the subject. The ocular pathology may comprise but is not limited to, for example any one or a combination of the following: open angle glaucoma, closed angle glaucoma, secondary glaucoma, pigmentary glaucoma, pseudoexfoliation glaucoma, uveitic glaucoma, neovascular glaucoma, low tension glaucoma and other glaucoma which either have a currently known or a currently unrecognized etiology.

A treatment decision may be based on the prognosis, monitoring or assessment of current properties of the entire aqueous outflow system tissues or regions of the tissue conducted in accordance with the measurement calculated with reference to FIG. 1. For example, a treatment may be based on the global or regional behaviors or properties of the tissues. Behaviors of the tissues may include tissue motion as measured in accordance with the system and method of FIG. 1.

FIGS. 2a-2g depict an example analysis of an ocular tissue, namely, the TM region of a subject. The FIGS. 2a-2g may be generated by a system such as the system 100 in FIG. 1, in combination with the calculations described above.

FIG. 2a depicts an example structural cross-sectional image 200 of the corneo-scleral limbus. The image 200 is on the micron scale and was taken from a subject in vivo. In the image 200, the cornea, sclera, collector channel (CC), Schlemm's canal (SC), trabecular meshwork (TM), ciliary body (CB), and iris are shown. The collector channel (CC), Schlemm's canal (SC), and trabecular meshwork (TM) are regarded as the aqueous humor outflow pathway in this Figure. An enlarged portion is shown depicting in further detail Schlemm's canal (SC) and the trabecular meshwork (TM).

FIG. 2b depicts an example velocity cross-sectional image 210 corresponding to the structural image 200 of FIG. 2a. In FIG. 2b, the trabecular meshwork region (TM) is shown.

Example instantaneous velocity waves of the non-TM tissue and the TM tissue are respectively plotted over time in graphs 220 and 230 in FIGS. 2c and 2d, respectively, together with a simultaneously recorded digital pulse wave. The instantaneous velocity waves and the simultaneous digital pulse wave may be calculated as described above with reference to FIG. 1. As can be seen in FIGS. 2c and 2d, compared with the non-TM velocity wave, the TM velocity wave qualitatively exhibits a periodicity with the same rhythm as the digital pulse wave (i.e., the heart rate).

FIG. 2e depicts a graph 240 illustrating exemplary power spectrums for each of the TM velocity wave and the digital pulse wave of FIG. 2d. In FIG. 240, the amplitude is plotted over time. As shown in FIG. 2e, the energy of both pulsatile waves is mainly concentrated in the same frequency components, i.e., the several harmonic frequencies, shown at 242 (1^{st}), 244 (2^{d}), 246 (3^{d}), and 248 (4^{th}), for example.

FIG. 2f depicts a graph 250 illustrating the significant correlation of the time instant between the TM pulse peaks and the digital pulse peaks of the velocity wave and the digital pulse wave of FIG. 2d. In FIG. 2f, TM f-time is plotted over digital p-time. The R² value is 0.998, and the P value is <0.0001. Thus, FIG. 2f demonstrates the temporal synchronization between the heart beating and the TM motion, but involves a phase lag.

FIG. 2g depicts a graph 260 illustrating the significant correlation of the frequency components between the TM velocity wave and the digital pulse wave of FIG. 2d. In FIG. 2g, the TM frequency is plotted over the digital frequency. The R² value is 0.996 and the P value is <0.0001.

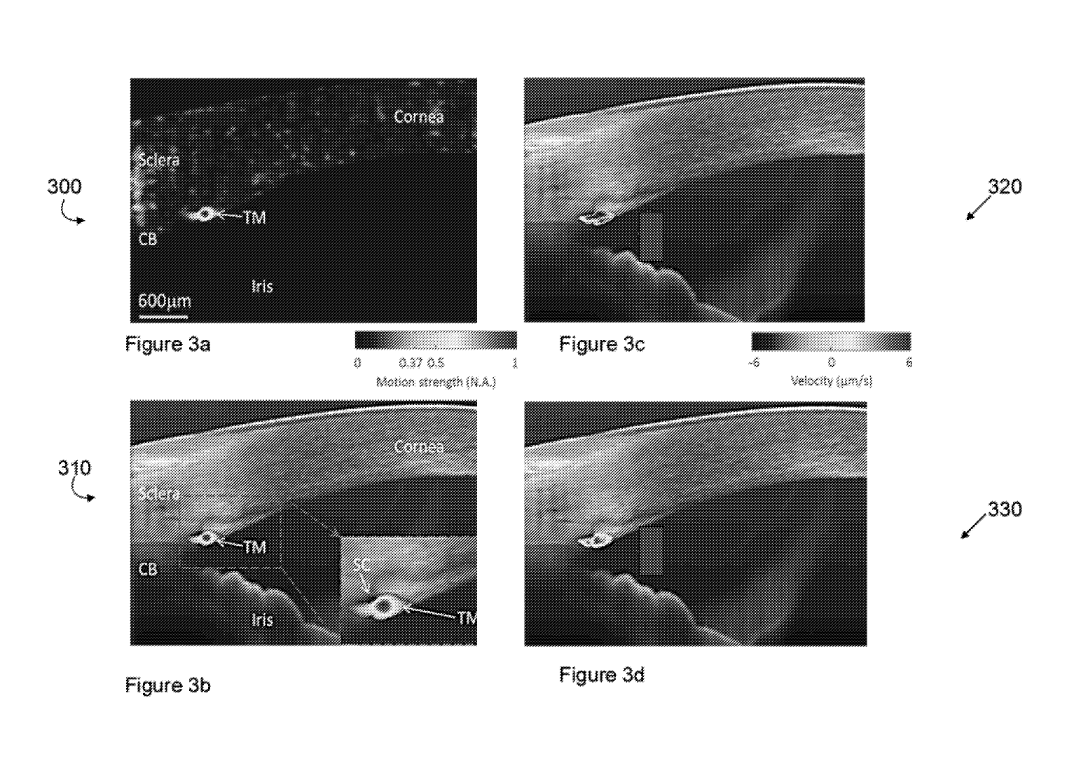

FIG. 3a depicts an exemplary normalized strength mapping 300 of the ocular tissue motion around the corneo-scleral limbus generated according to Equations 6-8.

FIG. 3b depicts isolated TM strength mapping superimposed with a corresponding structural cross-section image 310, generated by a system described with reference to FIG. 1. The enlarged portion of FIG. 3b suggests that the strongest motion center is located in the area under Schlemm's canal and then damped gradually from the center out to the periphery.

FIG. 3c depicts the isolated TM velocity cross-section superimposed with the structural cross-section 320, respectively corresponding to the downward velocity relative to the OCT probe beam.

FIG. 3d depicts the isolated TM velocity cross-section superimposed with the structural cross-section 330, respectively corresponding to the upward velocity relative to the OCT probe beam.

Thus, an OCT system integrated with a digital pulsimeter providing a synchronized digital pulse and a phase compensation algorithm that removes bulk motion allows for the quantitative characterization of the pulsatile tissue motion.

In an alternative embodiment, instead of using the digital pulse from a pulsimeter as the time reference to characterize the pulsatile tissue motion, the velocity pulse of the central retinal artery may be captured using the SD-OCT optimized for imaging of the retina and choroid.

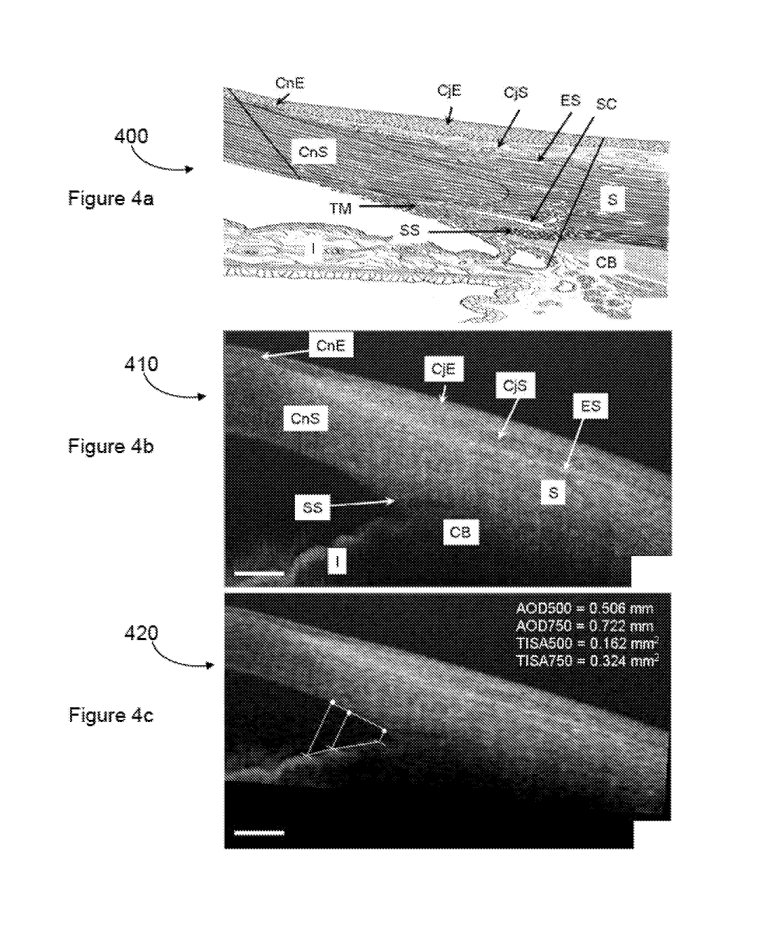

FIG. 4a depicts a schematic diagram 400 of the microstructural components in the limbal area of the eye. In FIG. 4a, the corneal epithelium (CnE), the corneal stroma (CnS), the conjunctival epithelium (CjE), the conjunctival stroma (CjS), the episclera (ES), the ciliary body (CB), the sclera (S), the scleral spur (SS), the trabecular meshwork (TM), and the Schlemm's canal (SC) are all depicted.

FIG. 4b depicts a structural cross-sectional image 410 of the human corneo-scleral limbus acquired from a temporal location, generated by an OCT system. In the corneal region, the corneal epithelium (CnE) and the corneal stroma (CnS) can be identified.

In one example embodiment, optical distortions in the images may be corrected using an algorithm based on Fermat's principle. The distortions may affect the microstructural images and may be due to a non-telecentric scan (i.e., the fan scan pattern) and the refraction of the probing light beam, for example.

FIG. 4c depicts a structural cross-section image 420 of the human corneo-scleral limbus acquired from a temporal location of FIG. 4b, after the correction of the non-telecentric and refraction distortion. Changes between FIGS. 4b and 4c can be observed in the width of the cornea and the anterior chamber angle, which can be primarily ascribed to the refraction distortion. In the example illustrated in the images in FIGS. 4b and 4c, a long focal length of about 75 mm of the objective lens in the OCT system was used. Thus, the non-telecentric distortion was not as obvious in FIGS. 4b and 4c. The angle opening distance (AOD) and the trabecular-iris space area (TISA) measurements were obtained and are shown in FIG. 4c to demonstrate the applicability for biometric measurements.

Images may be reconstructed by a computing system, such as the computing system 150 of FIG. 1, from the information provided in FIGS. 2a-2g.

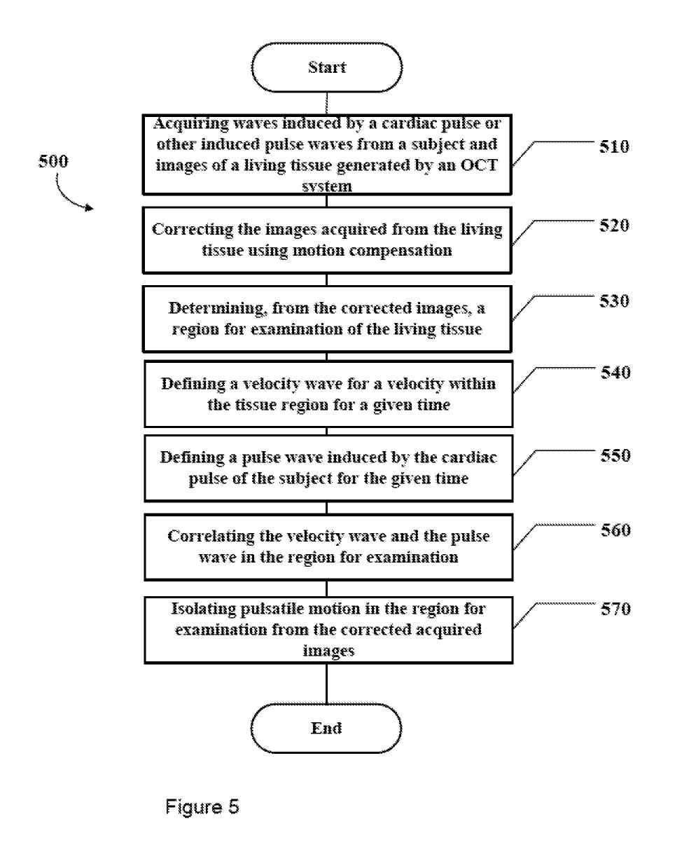

FIG. 5 depicts a simplified flow diagram of an example method that may be carried out to measure tissue motion within a living tissue, in accordance with at least one embodiment. Method 500 shown in FIG. 5 presents an embodiment of a method that, for example, could be used with the system 100.

In addition, for the method 500 and other processes and methods disclosed herein, the flowchart shows functionality and operation of one possible implementation of the present embodiments. In this regard, each block may represent a module, a segment, or a portion of program code, which includes one or more instructions executable by a processor for implementing specific logical functions or steps in the process. The program code may be stored on any type of computer readable medium, for example, such as a storage device including a disk or hard drive. The computer readable medium may include a physical and/or non-transitory computer readable medium, for example, such as computer-readable media that stores data for short periods of time like register memory, processor cache and Random Access Memory (RAM). The computer readable medium may also include non-transitory media, such as secondary or persistent long term storage, like read only memory (ROM), optical or magnetic disks, compact-disc read only memory (CD-ROM), for example. The computer readable media may also be any other volatile or non-volatile storage systems. The computer readable medium may be considered a computer readable storage medium, a tangible storage device, or other article of manufacture, for example. Alternatively, program code, instructions, and/or data structures may be transmitted via a communications network via a propagated signal on a propagation medium (e.g., electromagnetic wave(s), sound wave(s), etc.).

The method 500 allows for extracting tissue motion from a plurality of images acquired from the living tissue using an OCT system. The OCT system may be the same or similar to the system 100 of FIG. 1. The method 500 may be used to diagnose, develop a prognosis, or monitor treatment for a disorder of the living tissue.

Initially, the method 500 includes acquiring waves induced by a cardiac pulse or other induced pulse waves from the subject and images generated by the OCT system, at block 510. A computing system, such as the computing system 150, may send simultaneous trigger signals to each of the OCT system and the digital pulsimeter to acquire images of the subject and a cardiac pulse of the subject, respectively.

The method 500 then includes correcting the images acquired from the living tissue using motion compensation, at block 520. In one example embodiment, a cross-correlation method may be used to correct the images.

The method 500 includes determining, from the corrected images, a region for examination of the living tissue, at block 530.

The method 500 includes defining a velocity wave for a velocity within the tissue region for a given time, at block 540. The velocity wave may be defined as described with reference to FIG. 1 and Equation 1.

The method 500 includes defining a pulse wave induced by the cardiac pulse of the subject for the given time, at block 550. The pulse wave may be defined as described with reference to FIG. 1 and Equations 2 and 3.

The method 500 includes correlating the velocity wave and the pulse wave in the region for examination, at block 560. The correlation may be such as described with reference to FIG. 1 and the description regarding Equations 4 and 5.

The method 500 includes isolating pulsatile motion in the region for examination from the corrected acquired images, at block 570. The isolation may be such as described with reference to FIG. 1 and the description regarding Equations 6-8 coupled with the description regarding applying a motion mask.

The computing system 150 may plot the results, as described with reference to FIGS. 2a-g, and may use the information to reconstruct cross-sectional images of the living tissue, such as those described with reference to FIGS. 3a-4c.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims, along with the full scope of equivalents to which such claims are entitled. It is also to be understood that the terminology used herein is for the purpose of describing particular embodiments only, and is not intended to be limiting.

We claim:

1. A method of measuring tissue motion within a living tissue of an anterior segment and an aqueous outflow system of an eye in a subject comprising:
extracting tissue motion from a plurality of images acquired from the living tissue using an optical coherence tomography system, wherein the extracting comprises:
acquiring waves induced by a cardiac pulse or other induced pulse waves from the subject;
correcting the acquired images using motion compensation;
determining, from the corrected images, a region for examination of the living tissue;
defining a velocity wave for a velocity of motion within the region for examination for a given time;
defining a pulse wave induced by the cardiac pulse from the subject for the given time;
correlating the velocity wave and the pulse wave in the region for examination; and
isolating pulsatile motion in the region for examination from the corrected acquired images.

2. The method of claim 1, wherein the living tissue comprises an ocular tissue.

3. The method of claim 2, wherein the ocular tissue comprises an anterior segment eye tissue.

4. The method of claim 1, wherein measuring tissue motion within the living tissue of the anterior segment and the aqueous outflow system of the eye comprises measuring one or more of the following: measurement of tissue motion may include a measurement of one or more types of surface or internal tissue displacement and changes over time; surface or internal tissue motion and changes over time; trabecular tissue compliance and changes over time; trabecular tissue elasticity and changes over time; trabecular tissue velocity of movement and changes over time; trabecular tissue excursions and changes over time; elastic modulus of trabecular tissue and changes over time, diameter and volume of the juxtacanalicular space and changes over time; measurement of diameter and volume of the intertrabecular spaces and changes over time; movement of cellular or collagenous structures at the entrances or within collector channel ostia and changes over time; Schlemm canal diameter and volume and changes over time; collector channel ostia diameter and/or volume changes and changes over time; contour of the corneo-scleral junction and changes over time; angulation between the cornea and sclera at the corneoscleral junction and changes over time, diameter and/or volume of collector channels and changes over time; scleral spur position, orientation and changes over time.

5. The method of claim 1, wherein the method is used to diagnose, provide a prognosis, monitor treatment, or provide guidance in medical, laser or surgical management for a disorder of the living tissue of the aqueous outflow system of the eye.

6. The method of claim 1, wherein the subject is at risk of an ocular pathology or has an ocular pathology.

7. The method of claim 6 wherein the ocular pathology is glaucoma.

8. The method of claim 6, wherein the subject is at risk of an ocular pathology and the method comprises diagnosing whether the subject has an ocular pathology.

9. The method of claim 6, wherein the subject has an ocular pathology and the method comprises determining the likely rate of progression associated with the ocular pathology.

10. The method of claim 6, wherein the subject has an ocular pathology and the method comprises providing a prognosis for whether the subject is likely to respond to treatment for the ocular pathology.

11. The method of claim 6, wherein the subject has an ocular pathology and the method comprises monitoring efficacy of treatment of the subject for the ocular pathology.

12. The method of claim 10, further comprising making a treatment decision based on the prognosis or the monitoring.

13. The method of claim 9, further comprising making a treatment decision based on the measured tissue motion.

14. The method of claim 1, wherein the subject is a human subject.

15. The method of claim 1, further comprising:
mapping the extracted tissue motion into the extracted microstructural images of the living tissue.

16. The method of claim 1, wherein acquiring waves induced by the cardiac pulse or the other induced pulse from the subject and the images acquired by the optical coherence tomography system comprises simultaneously acquiring the waves induced by the cardiac pulse or the other induced pulse from the subject and the images acquired by the optical coherence tomography system by sending a trigger signal to the optical coherence tomography system and the digital pulsimeter.

17. The method of claim 1, wherein acquiring waves induced by the cardiac pulse comprises the optical coherence tomography system capturing a velocity pulse of a central retinal artery of the subject while simultaneously acquiring the images by the optical coherence tomography system.

18. The method of claim 1, wherein the images are acquired using the optical coherence tomography system by a method comprising:
applying light from a low coherence light source with a central wavelength of about 850-1800 nm through an optical coupler that splits light from the light source to the living tissue and to a mirror;
recombining light reflected from the living tissue and the mirror through the optical coupler; and
sending the recombined reflected light through a diffraction grating to a spectrometer.

19. A system for measuring tissue motion within a living tissue comprising:
an optical coherence tomography probe;
an optical circulator;
a coupler;
a spectrometer;
a digital pulsimeter; and
a physical computer-readable storage medium;
wherein the system acquires images from the living tissue,
wherein the physical computer-readable storage medium has stored thereon instructions executable by a device to cause the device to perform functions to extract tissue motion from the acquired images, the functions comprising:
acquiring waves induced by a cardiac pulse or other induced pulse from the subject;
correcting the acquired images using motion compensation;
determining, from the corrected images, a region for examination of the living tissue;
defining a velocity wave for a velocity of motion within the region for examination for a given time;
defining a pulse wave induced by the cardiac pulse or other induced pulse from the subject for the given time;
correlating the velocity wave and the pulse wave in the region for examination; and
isolating pulsatile motion in the region for examination from the corrected acquired images.

20. The system of claim 19, wherein the system for measuring tissue motion within the anterior segment and the aqueous outflow system of the eye comprises measurement of one or more types of surface or internal tissue displacement and changes over time; surface or internal tissue motion and changes over time; trabecular tissue compliance and changes over time; trabecular tissue elasticity and changes over time; trabecular tissue velocity of movement and changes over time; trabecular tissue excursions and changes over time; elastic modulus of trabecular tissue and changes over time, diameter and volume of the juxtacanalicular space and changes over time; measurement of diameter and volume of the intertrabecular spaces and changes over time; movement of cellular or collagenous structures at the entrances or within collector channel ostia and changes over time; Schlemm's canal diameter and volume and changes over time; collector channel ostia diameter and/or volume changes and changes over time; contour of the corneo-scleral junction and changes over time; angulation between the cornea and sclera at the corneoscleral junction and changes over time, diameter and/or volume of collector channels and changes over time; scleral spur position, orientation and changes over time.

21. The system of claim 19, wherein the functions of the physical computer-readable storage medium are further executable to map the extracted tissue motion into microvascular images of the living tissue.

22. The system of claim 19, wherein the functions of the physical computer-readable storage medium are further executable to map the extracted tissue motion into the extracted microstructural images of the living tissue.
